(19) United States
(12) Patent Application Publication
(10) Pub. No.: US 2011/0123451 A1
(43) Pub. Date: May 26, 2011

(54) Method for Cell Delivery Using Targeted Liposomes Associated with a Hemolysin

(76) Inventor: Max Kullberg, Aurora, CO (US)

(21) Appl. No.: 12/950,207

(22) Filed: Nov. 19, 2010

Related U.S. Application Data
(60) Provisional application No. 61/263,361, filed on Nov. 21, 2009.

(51) Int. Cl. A61K 49/00; A61K 9/127; A61K 39/395; A61K 38/16; A61K 38/22; A61K 38/14; A61K 31/70; A61K 31/7088; A61K 38/43; A61K 38/19; A61P 35/00; A61P 31/12

(52) U.S. Cl. 424/9.1; 424/450; 424/130.1; 424/158.1; 514/1.1; 514/9.7; 514/20.9; 514/23; 514/44 R; 424/94.1

(57) ABSTRACT

This invention describes a method for delivery of a therapeutic agent to target cells. Liposome delivery vesicles binds to a specific cell population via a targeting molecule attached to the liposome surface. After the liposomes bind to a target cell they are internalized into compartments within the cell called endosomes. It has been shown by prior art that by encapsulating a pore forming bacterial hemolysin into the lumen of a liposome, the endosome is broken down and drug is delivered into the cell cytoplasm. Instead of encapsulating the hemolysin within the liposome, this invention improves on current techniques by associating the hemolysin with the lipid membrane of the liposome. This modification reduces development cost significantly, eases production methods and increases the effectiveness and versatility of the treatment. Using this technique, we have demonstrated effective targeting and killing of Her-2 overexpressing tumor cells.

### FIELD OF THE INVENTION

This invention regards drug delivery to the cytoplasm of targeted cells.

### BACKGROUND

Overexpression of Her-2 occurs in 20-25 percent of breast cancers and typically corresponds with an aggressive and metastatic tumor (Engel and Kaklamani 2007, Azambuja et al. 2008). Her-2 is located in the cell plasma membrane and can be easily targeted with commercially available antibodies, making the receptor a logical target for chemotherapies (Johnston et al. 2006). After antibodies bind to Her-2, antibody-receptor complexes are clustered and internalized into cell endosomes, providing an opportunity for selective drug delivery into the cell cytoplasm (Park et al. 2001; Wartlick et al. 2004; Yang et al. 2007).

We have recently developed a liposome drug delivery system in order to target mammary epithelial cells that overexpress Her-2 (Kullberg et al. 2009, Kullberg et al. 2010). These liposomes are coupled with a pore forming bacterial hemolysin that compromises the target cell endosomes and delivers the contents of the liposome directly into the cellular cytoplasm of Her-2 overexpressing cells (FIGS. 1-4). The system delivers a 7-fold higher concentration of fluorescent marker to cells overexpressing Her-2 than to cells with normal Her-2 expression (FIG. 5), and demonstrates selective killing of Her-2 overexpressing cells when liposomes are loaded with a chemotherapeutic agent (FIG. 6).

Liposomes that are conjugated to Her-2 antibody are internalized into cells after binding to the Her-2 receptor on the cell surface. A liposome delivery systems that can penetrate the endosomal membrane and deliver drug directly to the cellular cytoplasm is of growing importance as the number of therapeutic macromolecules increases (El-Sayed et al. 2009). Currently, RNAi, gene therapy and cytotoxic peptides offer sophisticated alternatives to the traditional small molecule chemotherapy, but without an effective means of delivery to the cytoplasm, their therapeutic benefits will remain unrealized (Belting et al. 2005). After endocytosis, the macromolecules are exposed to the low pH environment of the endosome and eventually to the degradative enzymes in the lysosome unless they are supplied with a method for endosomal escape. Encapsulating these therapeutic macromolecules within liposomes that target cancer cells is an effective way of achieving large concentrations of the macromolecule within celluar endosomes (Amiji 2007). The liposomes protect the encapsulated package and preferentially localizes in the tumor tissue due to the leaky vasculature common in almost all solid tumors (Torchilin 2000, Maeda et al. 2008). However, methods are limited for cytoplasmic delivery of the encapsulated macromolecule after liposomes are endocytosed by the cell (Fretz et al. 2007, Xu et al. 2008).

In a previous study, we developed a technique using a pore forming protein, LLO, that was a modification of a successful method for penetrating the celluar endosome (Lee et al. 1996). We attached LLO to the outside of the liposomes and after being endocytosed with the liposomes, LLO forms pores in the endosomal membrane through which macromolecules can travel to reach the cytoplasm. Using this method with Her-2 targeting liposomes, we were able to target a fluorescent marker to the cellular cytoplasm of Her-2 overexpressing cells with high specificity (Kullberg et al 2010). When the fluorescent marker was replaced with a chemotherapeutic agent we achieved specific cytotoxicity for Her-2 overexpressing cells in vitro.

### SUMMARY OF THE INVENTION

The therapy that we have designed combines two liposome technologies in order to deliver drug directly to the cytoplasm of Her-2 overexpressing cells:

1. Cell Targeting: In the preferred embodiment of this invention, Her-2 specific antibody is conjugated to the liposome surface, allowing the liposomes to attach with great specificity to Her-2 expressing cells (Kullberg et al. 2009). Once bound to the cells, the liposome-receptor complexes are engulfed into endosomal compartments. Although this therapy has been designed to target Her-2 overexpression, the invention also describes targeting of other surface markers using established targeting molecules. A requirement is that when liposomes are targeted to the surface protein they are subsequently internalized into the cell, which greatly limits the number of applicable targets.
2. Cytoplasmic Delivery using LLO: To deliver directly into the cytoplasm of Her-2 overexpressing cells we utilize a pore forming bacterial hemolysin (Kullberg et al. 2010). Hemolysin is added to the liposome system after the liposomes have been formed and conjugated with Her-2 specific antibody. The hemolysin has an ability to associate with membranes and incorporates itself into the liposome membrane. We also find that the addition of cholesterol helps to strengthen this interaction between liposome and hemolysin. The present invention describes the addition of hemolyin to the membrane of targeted liposomes both with and without the addition of cholesterol.

### Brief Comparison of Invention to Prior Art

This invention is an improvement on prior art because it lowers production costs of hemolysin carrying liposomes considerably and creates a more effective delivery system. For a detailed description of how this invention improves on prior art please see the Detailed Description of Invention. Briefly, this invention reduces the amount of hemolysin protein necessary for production by 62 fold. By market prices, this reduces cost of liposome preparation from $4800 to $77 for 1 ml of liposomes. The second advantage is that this system does not necessitate breakdown of the liposome within a cell. With the prior art, U.S. Pat. No. 5,643,599, the hemolysin is in the lumen of the liposome and requires liposome breakdown so that the hemolysin can have access to the endosome membrane. With this invention, the hemolysin is attached to the liposome membrane where it can transfer to the endosomal membrane. Since the liposomes can remain intact but still transfer hemolysin to the endosome membrane, the techniques described in this invention can be used to easily incorporate hemolysin into already developed targeted liposome delivery systems.

### BRIEF DESCRIPTION OF THE DRAWINGS

FIG. 1: The figure shows a liposome with hemolysin attached to the lipid membrane. The example liposome shown has a therapeutic agent, X, that can be associated with the membrane or lumen of the liposome. The example liposome also is attached to a targeting molecule.

FIG. 2: The figure shows a liposome similar to FIG. 1 but with hemolysin incorporated into the lipid membrane.

FIG. 3: The figure shows a liposome similar to FIG. 1 but with hemolysin-cholesterol attached to the lipid membrane.

FIG. 4: The figure shows a liposome similar to FIG. 1 but with hemolysin-cholesterol incorporated into the lipid membrane.

FIG. 5: Delivery of calcein from LLO-liposomes to the cytoplasm after extended incubation at 37° C. A, B and C show representative photographs of cytoplasmic delivery to Her-2 overexpressing cells after a 1.5 hour incubation with LLO-liposomes and a total incubation time of 1.5 hr (A), 4 hr (B) or 10 hr (C) at 37° C. Likewise, D, E and F show normal cells exposed to the same conditions at 1.5 hr (D), 4 hr (E) or 10 hr (F). Analysis of the data indicates that at 10 hours, Her-2 overexpressing cells have a cytoplasmic calcein concentration that is over 7-fold higher than that in normal cells. Images were taken at a magnification of 200×.

FIG. 6: MTSV1-7 and ce2 cells treated with 100 nm gelonin-LLO liposomes. The 100 nm gel-LLO liposomes caused significant damage to Her-2 overexpressing cells (B), while Her-2 overexpressing cells treated with control LLO-liposomes remain unharmed (A). Normal cells treated with the gelonin-LLO-liposomes (D) are also damaged slightly by the treatment compared to control normal cells (C). However, analysis of the experiment performed in triplicate shows that the reduced viability of MTSV1-7 cells is significantly less than the ce2 cells. Images were taken at a magnification of 50×.

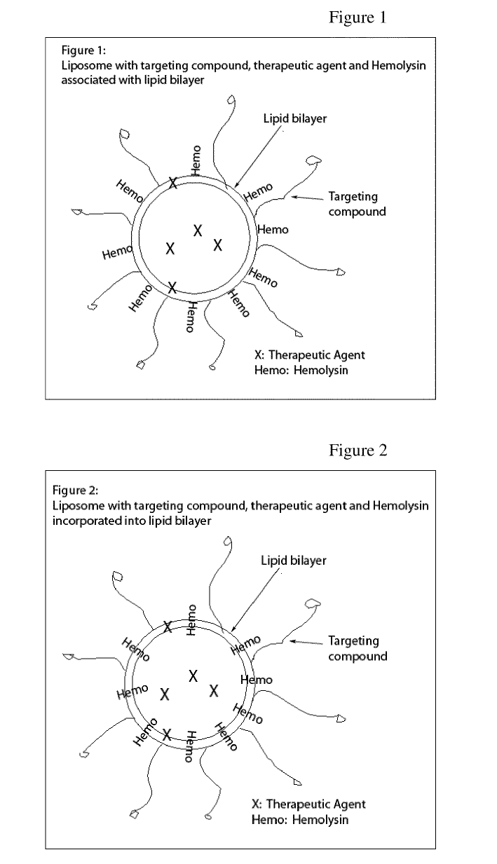

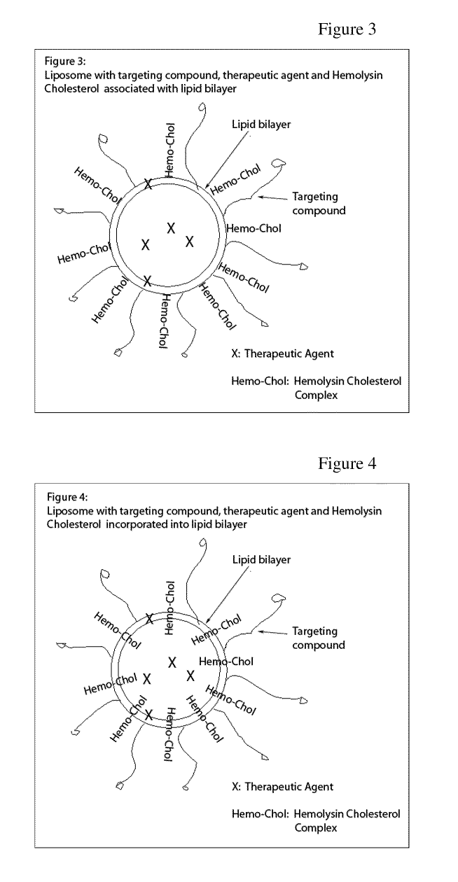

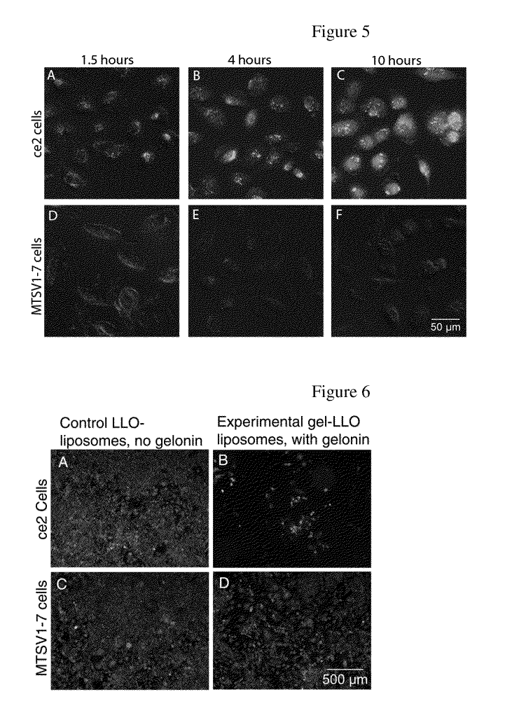

### DETAILED DESCRIPTION OF THE INVENTION

The invention involves the attachment of hemolysin to the membrane of a liposome that carries a therapeutic agent and is attached to a targeting molecule.

### Comparison of Invention to Prior Art

Lee et al. showed that when encapsulated in the lumen of pH-sensitive liposomes, a hemolysin formed pores in the endosomal membrane, increasing cytoplasmic delivery. This technique has been patented by Lee et al. 1996, U.S. Pat. No. 5,643,599. Our technique improves on this prior art because of how the hemolysin is associated with the liposome. Instead of being encapsulated within the lumen of the liposome as described by Lee et al., with this invention, the hemolysin is associated with the lipid membrane of the liposome either conjugated to the outside of the liposome or incorporated into the lipid membrane bilayer. This provides an advantage to the prior art in two regards. First, the liposomes are simpler and more cost effective to produce since they use a much smaller quantity of protein. Second, since the hemolysin is associated with the membrane it can easily transfer from the liposome to the endosome, allowing for more effective delivery and versatile application. We will discuss these two advantages in more detail.

### Cost Effective Production:

In the current invention, a small aliquot of hemolysin is added to liposomes after the liposomes are formed and a targeting molecule is attached. With the prior art, hemolysin is added at a concentration of 250 μg/ml (Lee et al. 1995). A large quantity of hemolysin is needed because the liposomes are forming around the hemolysin protein. The number of protein molecules within each liposome is dependent on the concentration of hemolysin present. With this invention, we take advantage of the hemolysin's ability to conjugate to the outside of a lipid membrane. The liposomes are exposed to a small amount of the hemolysin which incorporates into the membrane of the liposome. To achieve adequate concentrations of hemolysin it is only necessary to use 4 μg/ml, a 62 fold reduction in quantity of protein compared to the prior art (Kullberg et al. 2010). The predominate hemolysin used in both the prior art and this invention is Listeriolysin O (LLO) which is sold on the market at $192 for 10 μg (Bio-World Incorporated). With our invention one milliliter of liposomes would have a protein cost of $77 compared to $4,800 using methods described in the prior art. This is a considerable cost advantage which makes a treatment using hemolysin more realistic. In addition to cost, production methods of this invention are more simple than the prior art. In the prior art, liposomes are formed in the presence of hemolysin. Most liposome production involves extruding liposomes down to nanometer sizes (Torchilin and Weissig 2003). If hemolysin is present during the production, the temperatures necessary for this extrusion will damage the hemolysin and render the therapy ineffective. Also, targeting antibodies are often attached through chemistry that binds the amine group of the antibody (Amiji 2007, Kullberg et al. 2010). This reaction occurs spontaneously after liposomes are formed and would be impossible to carry out in the presence of hemolysin which also has amine groups. The method we use for attaching antibody to our liposomes would not be possible using techniques from the prior art.

### More Effective Delivery:

After liposomes are internalized into a target cell, the hemolysin must transfer from the liposome to the membrane wall of the endosome. With the prior art, hemolysin is trapped within the lumen of the liposome. In order for the hemolysin to transfer to the endosome, the liposome must be broken down. The liposomes are eventually broken down in the lysosomes of the cells, but this environment will render the hemolysin ineffective. Lysosomes are like the garbage disposal of the cell and are designed to chew up proteins, lipids and other molecules that enter them. To be effective the liposomes must release the hemolysin before this harsh environment. Therefore, Lee et al. designed their system using ph-sensitive liposomes (Lee et al. 1996). Their liposomes open up in the low ph of the endosome before reaching the lysosomes and the released hemolysin transfers to the endosome membrane. This method will only be effective with ph-sensitive liposomes. However, the vast majority of potential targeted therapies are not made with ph-sensitive liposomes and therefore cannot benefit from the prior art which encapsulates hemolysin in the liposome lumen. A problem with using ph-sensitive liposomes is that they are quickly taken up by the immune system unless they are coated with a polymer such as polyethylene glycol (PEG) which helps disguise the liposomes (Torchilin and Weissig 2003). PEG removes the ph-sensitivity of ph-sensitive liposomes which has been a great hindrance to advancing the field (Momekova et al. 2010). With our invention, hemolysin is attached to the membrane of the liposome and transfers freely from the liposome surface to the endosome membrane once the liposome is internalized. Any targeted liposome system, with or without PEG, can be easily modified to incorporate the hemolysin in the liposome membrane. Well before reaching the damaging lysosomes, the hemolysin transfers from the liposome to the endosome where it forms pores in the membrane. It is not necessary for the liposome to break down since hemolysin is associated with the liposome membrane. After the hemolysin transfers to the endosome and forms pores in the membrane, therapeutic molecules can transfer through the pores to the cytoplasm. Both our system and most other targeted liposomes would not benefit from hemolysin being incorporated within the lumen of the liposome as is done in the prior art (Park et al. 2001, Kullberg et al. 2010). Lee et al. produced an exciting system when they incorporated hemolysin within ph-sensitive liposomes and demonstrated increased delivery to cells. However, this invention demonstrates a clear improvement that leads to lower cost, ease of production and a more versatile delivery system that can be incorporated into almost any targeted liposome therapy.

As used herein:

The term “liposome” refers to a vesicular membrane structure comprised of a natural or synthetic phospholipid membrane or membranes, and optionally other membrane components such as cholesterol and protein. The vesicle can have a unilamellar, oligolamellar, or multilamellar membrane.

The term “targeting molecule” refers to an agent that binds to a defined target cell population, such as tumor cells. Preferred targeting moieties useful in this regard include antibody and antibody fragments, peptides, cytokines, peptidomimetics and hormones. Proteins corresponding to known cell surface-receptors (including low density lipoproteins, transferrin, EGF and insulin), fibrinolytic enzymes, anti-HER2, platelet binding proteins such as annexins, and biological response modifiers (including interleukin, interferon, erythropoietin and colony-stimulating factor) are also preferred targeting moieties. Ligands suitable for use within the present invention include biotin, haptens, lectins, epitopes, dsDNA fragments, enzyme inhibitors and analogs and derivatives thereof. Oligonucleotides binding to cell surfaces are also included. Analogs of the above-listed targeting moieties that retain the capacity to bind to a defined target cell population may also be used within the claimed invention. In addition, synthetic targeting moieties may be designed.

The term “therapeutic agent” refers to toxins, anti-tumor agents, drugs and radionuclides. Preferred toxins include Russell's viper venom, activated Factor IX, activated Factor X, thrombin, phospholipase C, holotoxins, pertussis toxin, dodecandrin, Shiga toxin, cobra venom factor, ricin, ricin A chain, pseudomonas exotoxin, diphtheria toxin, bovine pancreatic ribonuclease, ribonucleases, angiogenin, trichosanthin, pokeweed antiviral protein, abrin, abrin A chain, gelonin, saporin, modeccin, modeccin A chain, viscumin, volkensin, tritin, barley toxin, or any portion of these toxins. Ribosomal inactivating proteins (RIPs), naturally occurring protein synthesis inhibitors that lack translocating and cell-binding ability, are also suitable for use herein. Extremely highly toxic toxins, such as palytoxin and the like, are also contemplated for use in the practice of the present invention. Preferred drugs suitable for use herein include conventional chemotherapeutics, such as vinblastine, doxorubicin, bleomycin, methotrexate, 5-fluorouracil, 6-thioguanine, cytarabine, cyclophosphamide and cisplatinum, as well as other conventional chemotherapeutics as described in Cancer: Principles and Practice of Oncology, 2d ed., V. T. DeVita, Jr., S. Hellman, S. A. Rosenberg, J. B. Lippincott Co., Philadelphia, Pa., 1985, Chapter 14.

The term hemolysin refers to any pore forming protein expressed by gram-positive bacteria. Some examples of hemolysins include lysteriolysinu O (LLO), streptolysin O (SLO) and perfringolysin O

### Liposome Delivery System

With this delivery system, liposomes are attached to a targeting molecule and carry a hemolysin that is associated with the lipid membrane. Liposomes may also carry a therapeutic molecule that is delivered to the target cells. As described below a therapeutic molecule may also be administered separately and have its entry into the cell cytoplasm enabled by the liposomes. The liposomes will circulate through the body and bind to the target cells via the targeting molecule. After binding, the liposomes will be internalized into the endosomes of the target cells. The hemolysin which is located on the membrane of the liposomes transfers to the endosome membrane and forms pores in the endosome (Kullberg et al. 2010). As the therapeutic agent leaks from the liposome it will travel through these pores and exert its cytotoxic effect on the target cells. Using Her-2 targeting liposomes that incorporate gelonin and are attached to LLO we have shown specific killing of Her-2 overexpressing breast tumor cells (FIG. 6).

In addition, we have recently used the liposomes to facilitate the entry of a chemotherapeutic agent that is administered seperately from the liposomes. The chemotherapeutic agent is taken up independently into the endosomes of the cells. The chemotherapeutic agent becomes much more effective if its entry into the cell is enabled by a hemolysin carrying liposome. When the chemotherapeutic agent collocalizes with the liposomes inside of target cell endosomes, the hemolysin breaks down the endosome membrane and the therapeutic agent kills the cell. As described in the claims, this invention includes systems where the therapeutic agent is carried by the liposomes and also systems where a therapeutic agent is adminstered seperately and its entry into the cellular cytoplasm is facillitated by the hemolysin carrying liposomes.

### Description of Preferred Embodiments

Hemolysin Protein—Hemolysins are proteins expressed by gram-positive bacteria. Some examples of hemolysins include lysteriolysin O (LLO), streptolysin O (SLO) and perfringolysin O (PFO). The hemolysin used in this example is LLO,

Preparation of liposomes—Liposomes can be formed by a variety of methods by people familiar with the art. In this example liposomes were prepared by the film hydration-extrusion method (10). There are many different lipids and an infinite variety of combinations that will form liposomes. In the present invention liposomes can be made up of any combination of lipids, cholesterol or additives. In this example, liposomes are made with lipids DPPC:MPPC:DPPG:DSPE-PEG(3400)-NHS at a molar ratio of 82:10:3.5:4. The lipids in chloroform are mixed and a thin lipid film was formed by drying the lipids under nitrogen at 46° C. for 2 hours. Lipids were hydrated in 1 ml of distilled water at 46° C. for 4 minutes and filtered through a 220 nm filter system using a gas-tight sample-Luer Lock syringe from Hamilton (Reno, USA).

Cell targeting—Liposomes can be targeted to a particular type of cell by conjugating the liposome with a targeting molecule that can bind to a structure or marker on the cell surface. The targeting molecule can be an antibody, antibody fragment, oligonucleotide, peptide, hormone, ligand, cytokine, peptidomimetic, protein carbohydrate, chemically modified protein, chemically modified nucleic acids, chemically modified carbohydrates or any other molecule that allows liposomes to be taken up by cells. In this preferred embodiment, a 0.5 ml aliquot of the antibody trastuzumab (Her-2 antibody) in water at a concentration of 1.5 mg/ml was added to the filtered lipid. The suspension was incubated at 46° C. for 10 minutes and left at 4° C. overnight to allow for conjugation of the trastuzumab through amine binding. Liposomes were then extruded 10 times through a 200 nm filter at 46° C. using an extruder from Eastern Scientific (New York, USA).

Conjugation of hemolysin to liposomes—liposomes are coupled with a hemolysin or a hemolysin-cholesterol complex by exposing the liposomes to hemolysin or a hemolysin-cholesterol complex. In this preferred embodiment an 8 μl aliquot of LLO (2.0 mg/ml storage buffer) was added to 0.4 ml of liposomes at a lipid concentration of 0.33 mg/ml. The mixture was left at room temperature for 10 min and then 8 μl of cholesterol (25 mg/ml in 200-proof ethanol) was added to the solution. In this step, the LLO or LLO-cholesterol complexes associate with the lipid bilayer of the liposome. The liposomes were shaken slowly for 30 min and then spun at 3800 g for 3 min to pellet the cholesterol. The supernatant containing liposomes was run over a CL-4B column one more time to remove any unconjugated LLO or cholesterol that remained.

Therapeutic or diagnostic agent—A therapeutic agent or diagnostic agent is included either in the lumen of the liposome, associated with the liposome membrane or administered separately from the liposomes. In this preferred embodiment liposomes are filled with the therapeutic agent, gelonin. Liposomes are mixed with gelonin at a concentration of 1 mg/ml and then heated 46° C. for 3 minutes to allow the gelonin to flow into the interior of the liposomes. Loaded liposomes are separated from unencapsulated gelonin by size exclusion chromatography using a CL-4B sepharose. After this column purification, liposomes are ready to be administered. Examples of other therapeutic agents include diagnostic agent, peptide, an oligonucleotide, a nucleic acid, an antibiotic, an antimicotic, an anti-viral agent, an anti-cancer agent, an enzyme, a chemotherapeutic drug or toxin.

### STATEMENT REGARDING FEDERALLY SPONSORED RESEARCH OR DEVELOPMENT

This research was conducted at the University of Alaska. Following the recommendation of the University's office of technology transfer, the rights to this invention have been signed over to Max Kullberg.

### REFERENCES

1. Amiji M M. 2007 Nanotechnology for Cancer Therapy, CRC Press, London.
2. Azambuja, E., Durbecq, V., Rosa, D D, Colozza M, Larsimont, D., Piccart-Gebhart, M., Cardoso, F. (2008). Ann Oncol 19, 223-232.
3. Belting, M., Sandgren S., and Wittrup, A. (2005) Adv. Drug. Deliv. Rev. 57, 505-527.
4. El-Sayed, A., Futaki, S., and Harashima, H. (2009) AAPS J. 11, 13-22.
5. Engel, R. H., Kaklamani, V. G. (2007) Drugs 67, 1329-1341.
6. Fretz M. M., Hogset A., Koning G. A., Jiskoot W., Storm G. (2007) Pharm Res, 24, 2040-47.
7. Johnston, J. B., Navaratnam, S., Pitz, M. W., Maniate, J. M., Wiechec, E., Baust, H., Gingerich, J., Skliris, G. P., Murphy, L. C., Los, M. (2006) Curr Med Chem 13, 3483-3492.
8. Kullberg, M., Mann, K., and Owens, J. L. (2009) J. Drug Target. 17, 98-107.
9. Kullberg, M., Owens, J. L., Mann, K. (2010) J. Drug Target. 18(4): 313-320.
10. Lee K. D., Oh Y. K., Portnoy D. A., Swanson J. A. (1996) J Biol Chem, 271, 7249-52.
11. Maeda H., Bharate G. Y., Daruwalla J. (2009) Eur J Pharm Biopharm. 71, 409-19.
12. Momekova D, Rangelov S, Lambov N. 2010. Mol Biol, 605, 527-44.
13. Park, J. W., Kirpotin, D. B., Hong, K., Shalaby, R., Shao, Y., Nielsen, U. B., Marks, J. D., Papahadjopoulos, D., Benz, C. C. (2001) J Control Release 74, 95-113.
14. Torchilin, V. P., Weissig, V. (2003) Liposomes. University Press. Oxford.
15. Wartlick, H., Michaelis, K., Balthasar, S., Strebhardt, K., Kreuter, J., Langer, K. (2004) J Drug Target 12, 461-71.
16. Xu H., Deng Y., Chen D., Hong W., Lu Y., Dong X. (2008) J Control Release, 130, 238-45.
17. Yang, T., Choi, M. K., Cui, F. D., Kim, J. S., Chung, S. J., Shim, C. K., Kim, D. D. (2007) J Control Release 120, 169-177.

What is claimed is:

1. A method for delivering a therapeutic agent to specific target cells using liposomes where:
(a) Liposomes are targeted to cells via a targeting molecule located at the liposome surface and
(b) Liposomes carry a bacterial hemolysin that is associated with the lipid membrane of said liposome.

2. The method of claim 1, wherein a therapeutic agent is carried by the liposomes, either in the lumen of the liposomes or associated with the liposome membrane.

3. The method of claim 1, wherein a therapeutic agent is administered separately from the liposomes and its entry into the cell cytoplasm is enabled by the liposomes described in claim 1.

4. The method of claim 1, wherein the targeting molecule of claim 1a is an antibody or antibody fragment specific for the Her-2 receptor.

5. The method of claim 1, wherein the targeting agent of claim 1a is an antibody, antibody fragment, oligonucleotide, peptide, hormone, ligand, cytokine, peptidomimetic, protein carbohydrate, chemically modified protein, chemically modified nucleic acid, or chemically modified carbohydrate that targets a known cell-surface protein.

6. The therapeutic agent of claim 2 wherein said therapeutic agent is a diagnostic agent, peptide, an oligonucleotide, a nucleic acid, an antibiotic, an antimicotic, an anti-viral agent, an anti-cancer agent, an enzyme, a chemotherapeutic drug or toxin.

7. The therapeutic agent of claim 3 wherein said therapeutic agent administered separately is a diagnostic agent, peptide, an oligonucleotide, a nucleic acid, an antibiotic, an antimicotic, an anti-viral agent, an anti-cancer agent, an enzyme, a chemotherapeutic drug, a toxin or a liposomal based therapeutic agent.

8. The method of claim 1, wherein the liposomes are made up of a lipid or any combination of lipids that include but are not limited to phosphatidylcholine, phosphatidylserine, phosphatidylinositol, phosphatidylglycerol, phosphatidic acid, monosialoganglioside (GM1), polyethylene glycolphosphatidylethanolamine (PEG-PE), cholesterylhemisuccinate, phosphatidylethanolamine, oleic acid and cholesterol.

9. The method of claim 1, wherein the hemolysin of claim 1c is a hemolytic bacterial protein or genetically modified hemolytic bacterial protein, examples of which include lysteriolysin O (LLO), streptolysin O (SLO) and perfringolysin O (PFO)
